(19) United States
(12) Patent Application Publication
(10) Pub. No.: US 2026/0335820 A1
(43) Pub. Date: Oct. 29, 2026

(54) PLASMA TREATMENT DEVICE FOR TREATING A GAS PHASE AND ASSOCIATED METHOD

(71) Applicant: PRODEA DEPOLLUTING, Six-Fours Les Plages (FR)

(72) Inventor: Béatrice DRAZENOVIC, OLLIOULES (FR)

(21) Appl. No.: 19/116,161

(22) PCT Filed: Sep. 26, 2023
(86) PCT No.: PCT/EP2023/076601

§ 371 Date: Mar. 27, 2025

(30) Foreign Application Priority Data
Sep. 28, 2022 (FR) 2209837

(51) Int. Cl. B01D 53/32; H05H 1/24

(52) U.S. Cl. CPC B01D 53/32; H05H 1/24; B01D 2257/708; B01D 2258/06; H05H 2245/10

(57) ABSTRACT

A device for treating a gas phase, includes an assembly of electrodes including an injection electrode and an electrode connected to a ground of the device, which electrodes are separated by a separator material, the assembly being configured such that, when a voltage is applied, a plasma is generated between the injection electrode and the electrode connected to the ground, the separator material acting as an electrical insulator when the separator material is subjected to a voltage lower than a threshold voltage, which voltage is referred to as the priming voltage, and as an electrical conductor for receiving a current when the separator material is subjected to a voltage higher than or equal to the priming voltage.

### TECHNICAL FIELD

The present invention relates to the field of devices for the treatment of a gas phase by non-thermal plasma. It finds particularly advantageous application in the field of air treatment, for example indoor air, and/or pollutant emissions, and/or odour nuisances.

### STATE OF THE ART

It is known that volatile organic compounds (abbreviated as VOC) have harmful effects on health, with, for example, irritation of the eyes and mucous membranes, respiratory tract, cardiac and nervous system disorders, headaches and nausea. Some VOCs are even recognised as carcinogenic and/or toxic for reproduction or mutagenic (so-called ‘CMR’ (for Carcinogenic-Mutagenic-Reprotoxic) compounds).

The risks presented by VOCs have led to numerous regulations, resulting in a complex set of measures to meet the standards. These standards aim at reducing VOCs and odours, and to ensure satisfactory biological air quality.

Beyond VOCs, other harmful species can have adverse effects on health, the environment or olfactory comfort. This is the case with micro-organisms such as bacteria, viruses, fungi and/or their spores.

There is therefore a need to develop a system for the purification/decontamination of gaseous phases likely to comprise harmful species such as indoor air or pollutant emissions from human activity, for example from industry.

Over the last years, the indoor air purification market has developed with the commercialization of equipment displaying indoor air purification properties in the form of autonomous apparatuses, as well as construction and decoration materials highlighting depolluting properties. There is no certification for indoor air purification devices, and some of them can lead to the formation of compounds that are potentially more harmful than VOCs initially present.

Today, filtration is the most commonly used technique in this field. HEPA filters stop suspended materials, including organic materials. These, however, whether attached to the filters or having settled in the ducts of the filtration systems, are a favoured place for the growth of micro-organisms. In addition, nano-size viruses are not captured by these filters.

Non-thermal plasma technology (so-called ‘cold’ plasma) has many advantages: it is an electrical and not chemical, consumable-free, non-selective, compact and modular (capable of treating flow rates ranging from a few cm³/h to tens of thousands of m³/h) technology. Cold plasma treatments are effective on a multitude of pollutants, VOCs but also microorganisms. Indeed, cold plasma destroys the DNA of these microorganisms without distinction of size and is therefore effective on these pathogenic elements. It is to be noted that this technology is very well adapted for highly diluted pollution (low concentrations of pollutants), which makes it a technology of choice for the treatment of odours (low concentrations but high nuisances).

Conventional cold plasma treatments, for example generated by dielectric barrier discharge (abbreviated as DBD), are energy-intensive and generate harmful compounds such as ozone, degradation by-products of the pollutants treated.

Document US 2005/0118079 A1 describes a gas purification device using surface electrodes comprising photocatalysts in order to improve efficiency of a non-thermal plasma. ANSES (the French Agency for Food, Environmental and Occupational Health & Safety) advises against the use of physical and chemical air treatment, however, as their effectiveness, especially with regard to viruses, has not been proven. Following a sometimes incomplete degradation of pollutants, they can, further, negatively impact indoor air quality through the formation of compounds potentially hazardous to health, including CMR chemical agents.

Document FR2918293 A1 describes a gas treatment unit comprising electrodes separated by photocatalyst layers.

Document EP2762170 A1 describes a gas treatment device combining activity of a photocatalyst and a cold surface plasma.

Document US 2020/0398245 A1, a plasma reactor for the conversion of hydrogen sulphide, comprising an inner electrode, an outer electrode, and a dielectric barrier between the electrodes. One object of the present invention is therefore to provide a device for improving treatment of a gas phase by plasma, in particular by cold plasma, and especially that of air.

Further objects, characteristics and advantages of the present invention will become apparent upon examining the following description and the accompanying drawings. It is understood that other advantages may be incorporated.

### SUMMARY

To achieve this objective, according to one aspect, there is provided a device for treating a gas phase comprising at least one assembly of electrodes comprising at least one so-called injection electrode and at least one other electrode connected to the ground of the device, separated by a so-called <<separator material>>, the assembly being configured so that, under the application of an electric voltage, a plasma is generated between the at least one injection electrode and the at least one electrode connected to the ground.

The separator material exhibits electrically insulating behaviour when subjected to an electric voltage below a threshold voltage, so-called ‘primer voltage’, and electrically conductive behaviour allowing a current to flow therethrough when the separator material is subjected to an electric voltage greater than or equal to the primer voltage.

The separator material thus allows access to two plasma generation regimes: a homogeneous regime (electric discharges generated when the material behaves as an insulator) and an erratic regime, also designated an energetic regime (electric discharges generated when the material behaves as a conductor), which corresponds to an energetic multi-filament regime. These two regimes may appear simultaneously or successively.

The erratic regime does not exist in conventional DBD devices. This regime, together with the homogeneous regime, makes it possible to perfect the cold plasma treatment by improving degradation of the compounds treated and limiting residual harmful degradation by-products.

This further makes it possible to degrade a larger set of pollutants than with a conventional DBD device.

The synergy between these two regimes allows for more efficient electric discharges, which improves the efficiency and quickness of the gas phase treatment. The device can thus be made more compact and with a simplified design.

The treatment of the gas phase is thus improved by the joint application of these two plasma regimes on the gas phase flowing in the device. Furthermore, the use of a catalyst is avoided, which simplifies the device as well as its maintenance.

Furthermore, during the development of the invention, it has been highlighted that this type of separator material makes it possible to lower value of the electric voltage to be applied to generate the plasma. The cost, especially the energy cost, of treating a gas phase is thus lowered. Further, the safety of use of the device is improved.

Another aspect relates to a method for treating a gas phase using the device according to the first aspect, and comprising:
introducing the gas phase into the at least one assembly of electrodes,
applying an electric voltage to the assembly so as to generate a plasma between the at least one injection electrode and the at least one electrode connected to the ground to treat the gas phase.

The method has the effects and advantages described relative to the device according to the first aspect.

Further, as the plasma discharges are more efficient, the treatment time of the gas phase can be reduced relative to treatment by a conventional DBD device. This is particularly advantageous for killing viruses, which generally requires more energy (and more time) than that of VOCs.

### BRIEF DESCRIPTION OF THE FIGURES

The purposes, objects, as well as characteristics and advantages of the invention will be better understood from the detailed description of one embodiment thereof which is illustrated by the following accompanying drawings wherein:

FIG. 1 represents an overview of the device and its principle of operation, according to one exemplary embodiment.

FIG. 2 shows a diagram of the electrical circuit for studying electrical behaviour of the separator material, according to one example.

FIG. 3 schematically represents the electrical behaviour of the separator material according to several exemplary embodiments, in comparison with an insulator of a DBD device.

FIGS. 4 to 6 represent different electrode geometries in an assembly, according to several exemplary embodiments.

FIGS. 7 to 9 represent different arrangements of assemblies, according to several exemplary embodiments.

FIGS. 10A and 10B represent two graphs showing the percentage of VOC degradation, respectively ethylene and isopropanol, as a function of the voltage applied to an assembly to generate cold plasma, in comparison to two assemblies of DBD devices of the state of the art.

The drawings are given by way of exemplary and not limiting purposes of the invention. They are schematic representations of principle for facilitating understanding of the invention and are not necessarily to the scale of practical applications. In particular, relative dimensions between the VOCs, the electrodes and the device are not representative of reality.

### DETAILED DESCRIPTION

Before beginning a detailed review of embodiments of the invention, optional characteristics are set out hereinbefore which may possibly be used in combination or alternatively.

According to one example, the primer voltage is between 1 kV and 10 kV, preferably between 3 kV and 6 kV. These breakdown voltage parameters are taken for an AC 50 Hz signal and for a point/point electrode geometry on the surface of the separator material, for a gap of between 5 and 15 mm.

According to one example, the separator material is selected from the group consisting of: a semiconductor material, for example a semiconductor ceramic, a semiconductor polymer, a composite material comprising conductive or semiconductive particles dispersed in a matrix. It should, however, possess dielectric properties with an insulating/conductive transition for the selected operating voltage range. The insulating phase also has the charge trapping/de-trapping characteristics required to allow the energetic regime. When the separator material is a composite material comprising conductive or semiconductive particles dispersed in a matrix, the separator material offers a variety of choices in the nature of the particles and the matrix, making it possible to obtain the two plasma generation regimes. This gives more flexibility in the nature of the material relative to a homogeneous material, especially for modulating thermal, mechanical and electrical properties. This is quite different from the materials of existing solutions, which on the one hand do not aim at achieving these two regimes, and on the other hand are homogeneous materials formed of a single phase or multi-layer assemblies of homogeneous materials. Especially, this is completely different from solutions implementing a photocatalytic layer of TiO₂ under UV irradiation. This type of coating has a high dielectric constant (typically greater than 6), and does not allow for obtaining an erratic regime.

According to one example, the conductive or semiconductive particles are homogeneously dispersed in the matrix. They can be distributed substantially throughout the thickness of the matrix, and more particularly substantially throughout the volume of the matrix.

According to one example, the matrix is insulating. The separator material can therefore be a composite material comprising an insulating matrix and conductive or semiconductive inclusions. The separator material is thus overall semiconductive and presents a microstructure.

The plasma primer process then no longer depends on the intrinsic properties of one of the constituents and the surface microstructure, but on the whole material. In the presence of conductive grains surrounded by insulating grain boundaries, the charges are trapped on the surface of the grains, thus inducing a curvature of the valence and conduction bands with the formation of a double Schottky barrier. The material is thus particularly adapted to access the two plasma generation regimes: the homogeneous regime (electric discharges generated when the separator material behaves as an insulator) and the erratic regime, also designated the energetic regime (electric discharges generated when the behaviour of the separator material is conductive). According to one example, the separator material being a composite material comprising conductive or semi-conductive particles dispersed in an insulating matrix, the insulating matrix is based on or made of a material selected from the group consisting of a polymer and a ceramic, and the conductive or semi-conductive particles are based on or made of a material selected from the group consisting of a metal, an intermetallic compound, a metal alloy or a ceramic.

According to one example, at least 90% of the thickness of the material separating the two electrodes, and preferably at least 95%, of this thickness is formed by the separator material. According to one example, the separator material has no additional coating.

According to one example, the device does not comprise any means of processing a gas phase other than the plasma generated with the assembly of electrodes.

According to one example, the separator material does not have any porosity. According to an alternative embodiment, the separator material has a non-zero mean porosity, preferably greater than or equal to 10%. Preferably this porosity is between 10% and 30%. This porosity in the material has the advantage of improving performance of the device. This porosity is open, with pores being present on the outer face of the material and therefore in contact with the environment surrounding the material. This porosity induces a gas adsorption effect on the separator material, which increases efficiency of the device. A porosity range between 10% and 30% is particularly advantageous for a homogeneous regime. According to one example, the separator material has a mean porosity of between 3 and 10%.

This range is more advantageous for increasing the life time in an erratic regime.

According to one example, the material is a semiconductor ceramic, including a microstructure comprising:
5 to 40% by volume of a particulate conductive phase,
60 to 95% by volume of a particulate insulating phase, the particle size of the conductive phase being between 5 nm and 11 μm, 65 to 80% of the conductive particles having a mean diameter of less than 1 μm and 20 to 35% of the conductive particles having a mean diameter of between 1 and 11 μm;
and the distance between two neighbouring conductive phase particles being between 30 Angstroms and 5 μm.

According to one example, the material constituting the conductive phase is selected from the group consisting of MoSi₂, TiB₂, TiN, Ni₃Si, HfB₂, ZrB₂.

According to one example, the particle size of the insulating phase is between 0.3 and 3 μm.

According to one example, the material constituting the insulating phase is selected from Al₂O₃, mullite, Si₃N₄.

According to one example, the ceramic comprises 15 to 25% by volume of MoSi₂, and preferably 21 to 24% by volume of MoSi₂.

According to one example, the conductive phase based on MoSi₂ particles further comprises between 0% and 2% by weight of carbon.

According to one example, the surface of the semiconductor ceramic is vitrified.

According to one example, the conductive phase based on MoSi₂ particles further comprises 1% by weight of an element selected from Al, Ta, Ti, Zr, Y and B.

According to one example, the ceramic further comprises 0.1 to 0.9% by weight of a lanthanide compound.

According to one example, the voltage applied to generate the plasma has a non-zero value of less than 10 kV and preferably 6 kV.

According to one example, the injection electrode has, on at least one portion of the assembly, a point configuration also designated a point electrode. The point configuration of the injection electrode promotes electric discharges in the erratic regime, in synergy with the nature of the separator material as well as the electric signal and the geometry of the electrodes.

According to one example, the device is more particularly configured to make the homogeneous and erratic discharge regimes coexist temporally or spatially during plasma generation.

According to one example, the separator material has a cylindrical shape extending along a main direction of extension of the assembly, and:
on a first portion of the assembly, the electrode connected to the ground and the injection electrode together form a coaxial structure on either side of the separator material, about the main direction of extension of the assembly,
on a second portion of the assembly, distinct from the first portion, the electrode connected to the ground extends coaxially to the main direction of extension of the assembly about the separator material, and the injection electrode has a point configuration disposed in an internal volume defined by the separator material.

On the first portion, the plasma is generated in the homogeneous regime. On the second portion, the plasma is mainly generated in the erratic regime. The assembly thus has two treatment zones in which the two regimes coexist and are spatially separated. The gaseous phase thus transits through the first portion and the second portion to be successively treated by discharges according to the two regimes, in order to maximise the degradation of the compounds to be treated.

According to one example, the first portion is disposed upstream of the second portion, along the direction of circulation of the gaseous phase in the assembly.

According to one example, the first portion and the second portion are separated by an intermediate portion, the assembly being configured so that under the application of an electric voltage, a plasma is generated only in the first and second portions. The intermediate portion makes it possible to increase spatial separation between the first and second portions. In this way, by-products generated by the plasma treatment in one of these portions, and preferably in the first portion, can react with each other before the treatment is completed in the other portion, preferably the second portion.

Depending on the volume restriction associated with the device, it can also be contemplated that the first and second portions are directly juxtaposed, in order to limit overall size of the device. According to one example, the separator material has a first face and a second face opposite to the first face, the injection electrode and the electrode connected to the ground are disposed on the first face of the separator material, the injection electrode and the electrode connected to the ground being disposed at a distance from each other. The assembly can thus have a planar geometry, the electrodes being disposed on a same face of the separator material. This geometry allows the generation of plasma discharges according to the two, homogeneous and erratic, regimes. These two regimes can coexist spatially. The assembly thus has a treatment zone in which the two regimes coexist and follow each other temporally. The gaseous phase thus transits through the treatment zone and is successively treated by discharges according to the two regimes, in order to maximise degradation of the compounds to be treated. According to one example, on the second face of the separator material, at least one injection electrode and at least one electrode connected to the ground are further disposed at a distance from each other. The planar geometry of the assembly thus allows the electrodes to make both faces of the separator material functionalised. Compactness of the device is further improved.

According to one example, the injection electrode and the electrode connected to the ground each have a point configuration facing between the injection electrode and the electrode connected to the ground. This promotes surface discharge rather than volume discharge.

According to one example, the electrode connected to the ground has a first face comprising at least one opening leaving the separator material apparent, at least one injection electrode being disposed in the at least one opening and extending from the separator material in an oblique direction, preferably perpendicular, to the first face of the electrode connected to the ground. This geometry can be described as a “point/surface plane”. One advantage of this geometry is that it promotes the amount of plasma generated.

According to one example, the assembly is connected to a power supply module configured to supply the assembly with a pulsed electric voltage signal. The pulsed signal is used to apply a pulsed voltage to generate the plasma. This type of current is compatible with the set of assembly geometries.

According to one example, the assembly is connected to a power supply module configured to supply the assembly with an alternating electric voltage signal. An alternating signal is cheaper and simpler to implement in use. The design of the device is therefore simplified and its cost reduced. For this, it is preferable that the electrodes are disposed on the same face of the separator material.

According to one example, the device comprises a plurality of assemblies stacked along at least one direction perpendicular to a main direction of extension of the assembly. The plurality of assemblies makes it possible to increase the amount of the gas phase treated, while reducing the dead volume between the assemblies to increase compactness of the device.

According to one example, the voltage applied to generate the plasma has a non-zero value of less than 10 kV, between 3 and 6 kV. The voltage applied to generate the plasma is thus reduced relative to that generally used in DBD devices, typically greater than or equal to 20 kV.

According to one example, in the device, the gas phase is at atmospheric pressure.

In the rest of the description, the term “on” does not necessarily mean “directly on”.

Thus, when it is stated that a piece or member A is bearing “on” a piece or member B, this does not mean that pieces or members A and B are necessarily in direct contact with each other. Pieces or members A and B may be in direct contact or may be bearing on one another by means of one or more other pieces. The same is true of other expressions such as, for example, ‘A acts on B’, which may mean that “A directly acts on B” or that “A acts on B via one or more other pieces”.

In the detailed description that follows, use could be made of terms such as “horizontal”, “vertical”, “longitudinal”, “transversal”, “upper”, “lower”, “top”, “bottom”, “front”, “rear”, “internal”, “external”. These terms should be interpreted in a relative manner in relation to the normal position of use of the device, and more particularly to the direction of circulation of the gas phase in the device and the assembly. For example, the notion of “longitudinal” corresponds to the main direction of extension of the assembly, a direction which is substantially parallel to the direction of circulation of the gas phase in the assembly.

A reference frame will also be used where the longitudinal or rear/front direction corresponds to the axis x, the transverse or right/left direction corresponds to the axis y and the vertical or bottom/top direction corresponds to the axis z.

It is set out that within the scope of the present invention, the thickness of a material is measured along a direction perpendicular to the surface where this material has its main extension. The thickness of the separator material can especially be taken along a direction perpendicular to the main faces of the separator material, at least one of these faces being in contact with at least one electrode of the device. It is therefore understood that the thickness of the material can be the smallest of the other dimensions of the material (relative to the length, width or radius for example).

The device 1 for treating a gas phase 2 is now described according to several exemplary embodiments with reference to the figures.

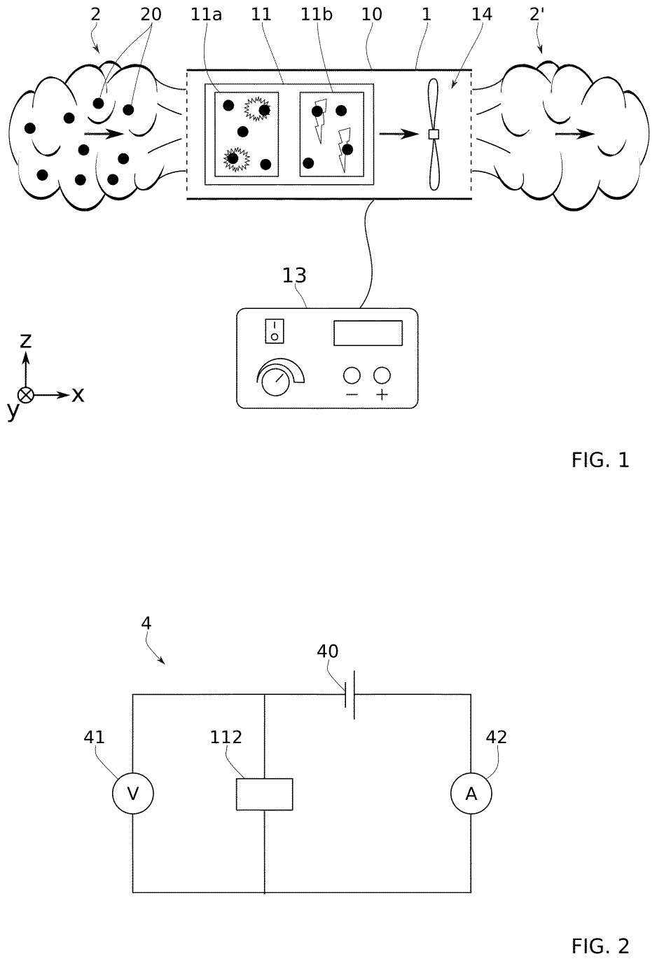

As illustrated in FIG. 1, the device 1 is configured such that a gas phase 2 to be treated flows in the device 1. The gaseous phase 2 may comprise suspended species 20, especially polluting species, for example VOCs, and/or microorganisms such as bacteria, fungi and/or their spores, viruses, particles. In order to degrade these species 20, the device 1 comprises a body 10 through which the gaseous phase 2 circulates. For this, the device may comprise a module 14 for sucking the gaseous phase 2, for example a fan. The device 1 therefore comprises an air circulation circuit from an inlet of the body 10 to an outlet of the body 10, not represented in the figures.

The device 1 comprises at least one assembly 11 of electrodes. Each assembly 11 comprises at least one injection electrode 110 and at least one electrode 111 connected to the ground 12 of the device 1, hereinafter designated as the electrode connected to the ground 111, as illustrated in FIGS. 4 to 6, subsequently described in more detail. The assembly 11 is configured so that, under the application of an electric voltage, a plasma is generated between the injection electrode 110 and the electrode connected to the ground 111. This voltage can be more particularly applied by a power supply module 13 connected to the electrodes 110, 111. The power supply module 13 can be configured to supply the device 1, and more particularly the assembly 11, with an electric current enabling this voltage to be applied.

During its circulation in the device 1, the gaseous phase 2 comprising the species 20 to be treated circulates in the assembly 11. The plasma 3 generated in the assembly acts on the species 20 causing their degradation. The gaseous phase 2′ at the outlet of the device 1 can thus be considered as depolluted or decontaminated, that is to say the amount of species 20 is at least reduced, preferably the species 20 are removed or killed.

The injection electrode 110 and the electrode connected to the ground 111 are separated by a separator material 112. In conventional DBD devices, this separator material 112 is an electrical insulator. In device 1, the separator material 112 exhibits electrically insulating behaviour when subjected to some electric voltage below a threshold voltage, hereinafter designated as the primer voltage Vo. When the separator material 112 is subjected to an electric voltage greater than or equal to the primer voltage Vo, it exhibits electrical conductive behaviour allowing an electric current to flow therethrough.

This separator material 112 therefore has a non-linear electrical resistance (the U/I ratio is not constant). Below the primer voltage Vo, it has a high impedance and therefore a very low leakage current, and preferably a zero leakage current. Above the primer voltage Vo, the material 112 has a lower impedance and allows a higher leakage current to flow therethrough. In other words, beyond the primer voltage Vo, the impedance of the separator material 112 drops to allow electrical conduction in the form of a leakage current. When the voltage returns to a level lower than the primer voltage, the impedance increases again to return to its initial value. The transition can be abrupt when changing from insulating/conductive behaviour but also from insulating/low leakage current/conductive behaviours. The wider the transition zone (i.e., voltage range), the more homogeneous the weakly mixed regime.

It is therefore understood that the separator material 112 can change from one behaviour to another and vice versa according to the applied voltage, unlike an insulator which is destroyed beyond its breakdown voltage and which can no longer return to insulating behaviour when the voltage drops.

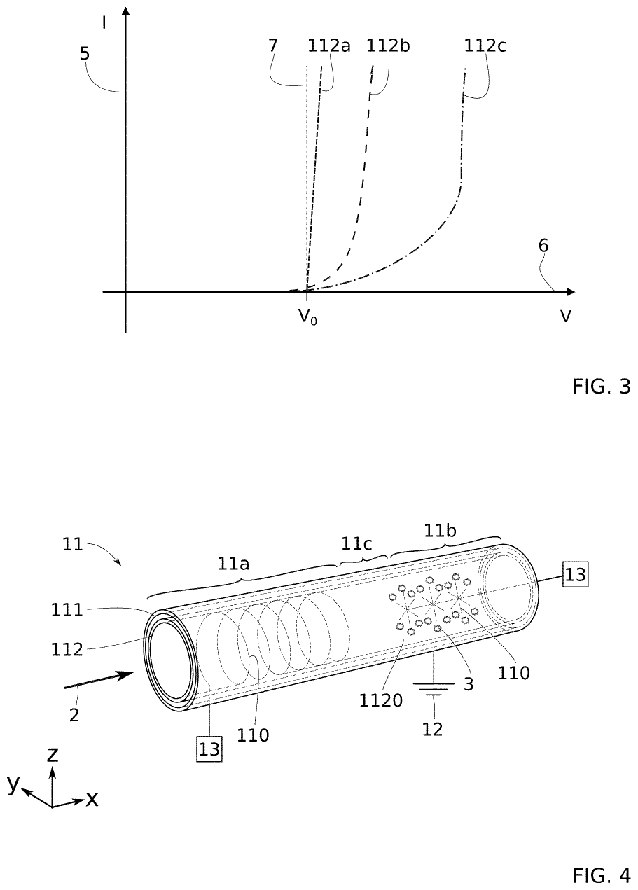

These electrical behaviours of the separator material 112 can be observed by virtue of mounting 4 illustrated in FIG. 2, in which a power source 40 supplies an electrical circuit connected to the separator material 112. The electrical circuit comprises a voltmeter 41 connected in parallel to the separator material 112 and an ammeter 42 connected in series to the circuit. This allows for the measurement of the voltage across the separator material 112 and the intensity of the current flowing therethrough. In this mounting 4, the material 112 can be held between two electrodes (for example, two solid brass discs) by a spring system coated with conductive paste for better electrical contact. FIG. 3 is a graph representing the intensity (I) of current 5 as a function of voltage 6 (V) across the separator material, for an insulator 7 and for three examples of separator material 112a, 112b, 112c.

At the breakdown voltage, the insulator 7 is destroyed and no longer exhibits insulating behaviour. When subjected to a voltage higher than the primer voltage Vo, the isolating materials 112a, 112b, 112c change from an insulating behaviour where the current I is very low or even zero, to a more conductive behaviour wherein the current I increases with the voltage across the material, a leakage current then being measurable. This increase may be sudden or more or less gradual, as illustrated by the three curves 112a, 112b, 112c.

The rate of this increase may influence nature of the different non-thermal plasma regimes generated during the use of device 1, including a homogeneous regime, a mixed regime or an erratic regime (also designated energetic regime).

The primer voltage can be between 1 kV and 10 kV, preferably between 3 kV and 6 kV.

This type of value allows for efficient and controlled operation to access the two plasma generation regimes, and especially in combination with an AC 50 Hz signal, an electroceramic material and a surface-type geometry.

In general, a person skilled in the art will be able to adapt the value of the primer voltage as a function of the nature of the signal, the nature of the material and the geometry for the targeted application.

The geometry especially defines the shape of the electrodes, the placement of the electrodes relative to the separator material, the interelectrode gap, the stacking or arrangement of the cells.

The skilled person will be able to adapt the variable parameters without any difficulty to obtain the desired effect. In particular, they will be able to adapt the nature of the signal (for example for a pulse signal: its shape, its rising front, its duration, its value), its frequency, the geometry and the placement of the electrodes (especially the distance, also designated as the inter-electrode “gap”). Also naturally, the reactor will be dimensioned relative to the desired end use in terms of flow rate.

By virtue of these electrical behaviours of the separator material 112, when an electric voltage is applied to the assembly 11, a cold plasma can be obtained by charges according to two main regimes: the homogeneous surface regime by discharge in the insulating behaviour or an energetic multi-filament volume regime by discharge in the conductive behaviour, or even a mixed regime.

The energetic regime is not accessible with insulating materials in conventional DBD devices. This regime, in synergy with the homogeneous regime, makes it possible to improve degradation of species 20 and to extend the number of species 20 that can be treated by plasma. For example, some VOCs are more effectively degraded by a homogeneous plasma, such as sulphides, aromatic compounds and halogenated VOCs. The treatment of VOCs in a homogeneous plasma can further generate ozone and/or harmful reaction by-products.

During the development of the invention, it has been observed that a treatment complementary to a homogeneous plasma 3 by an energetic plasma 3 makes it possible to limit, and preferably remove, residual ozone as well as reaction by-products, as well as to achieve better selectivity in CO₂. This complementary treatment further makes it possible to degrade molecules that would not have been degraded by a homogeneous regime plasma discharge 3 being of insufficient energy, especially ketones or even acids.

Furthermore, the minimum voltage to be applied to generate a plasma 3 is lower than for a conventional DBD device by virtue of the separator material 112. The discharges are also more efficient, thereby reducing degradation time of the species 20.

Furthermore, the coexistence of plasma discharges 3 in a homogeneous regime and plasma discharges 3 in an energetic regime allows for the aggregation of particles possibly present in gas phase 2, in the manner of an electrostatic filter. It is therefore possible to trap particles at the outlet of device 1, for example by adding a filter. Deposits inside device 1 are reduced, which limits fouling of assembly 11 and/or the risk of short-circuit.

Device 1 can be used in a method for treating a gas phase 2. This gas phase 2 can be indoor air, for example inside a building, or a gas phase from industrial waste.

Gas phase 2 can be introduced into assembly 11, for example by suction through the suction module 14. The suction module is dimensioned relative to the treatment capacity of the device, which is in turn dimensioned as a function of the treatment requirement of the gas phase. For example, for the treatment of the air in a 30 m² room, a flow rate of 250 Nm³/h is desirable.

Electric voltage can be applied to the assembly 11 to generate a plasma 3 between the injection electrode 110 and the electrode connected to the ground 111 to treat the gas phase 2. The voltage applied to generate the plasma 3 can have a non-zero value of less than 10 kV, for example between 3 and 6 kV.

According to one example, the gas phase 2 is continuously introduced into the device 1. The plasma treatment 3 can also be carried out continuously.

Alternatively, it can be contemplated that the plasma treatment is discontinuously or non-constantly carried out. For example, it can be contemplated that the activation of the treatment and/or its flow rate is servo-controlled as a function of pollution of the gas phase to be treated. For this, sensors arranged to measure a parameter relating to the pollution or contamination of the gas phase can be provided. The servo-control is a function of these measurements. Thus, as a function of this servo-control, the volume of gas phase 2 sucked in can then be treated by successive volumes.

### Particular Examples of Separator Material

The separator material 112 is now described according to several exemplary embodiments.

The separator material 112 can be selected from:
a semi-conductor material, for example a semi-conductor ceramic, such as for example SiC, ZnO or GaN,
a semi-conductor polymer,
a composite material comprising conductive or semi-conductive particles dispersed in an insulating matrix.

The composite material may, according to one example, comprise conductive or semiconductive particles dispersed in an insulating matrix. The insulating matrix may be based on or made of a polymer or a ceramic, for example. The conductive or semiconductive particles may be based on or made of a metal, a metal alloy or a ceramic. For example, the composite material may be polymer/metal, polymer/ceramic, cermet (ceramic/metal) or ceramic/intermetallic.

The separator material may have some porosity. The advantage of this porosity in the material is that it improves performance of the device. This porosity is preferably greater than or equal to 10%. Preferably, this porosity is between 10% and 30%.

It can also be contemplated that the porosity is zero or small.

When a volume plasma is generated, for example with one electrode in contact with the separator material 112 (for example, an electrode connected to the ground 111) and the other electrode (for example the injection electrode 110) not in contact with the material, being separated from this material 112 by a gas (air for example), the greater the thickness of the separator material 112, the greater the voltage required to prime the cold plasma. The lower this thickness, the more the erratic regime is favoured since it will appear more quickly for lower primer voltages. However, if this thickness is too thin, it limits the life time of the reactor due to excessive stresses on said material (for example, material erosion or mechanical stresses). Preferably, the thickness of the separator material 112 is between 0.5 mm and 15 mm, preferably between 2 mm and 6 mm. This thickness may be greater than or equal to 1 mm, preferably 2 mm, for example substantially equal to 3 mm.

When generating a surface plasma, it has been shown during the development of the invention that the thickness does not significantly influence the plasma. However, this thickness can impact the life time of the reactor. Preferably, the thickness of the separator material 112 is greater than or equal to 1 mm, preferably between 2 mm and 6 mm. According to one particular example, this thickness is approximately equal to 5 mm.

It will be noted that these thicknesses are correlated to the dimensioning of the reactor and can therefore be adapted as a function of these dimensions.

In the following, a particular example is described in a non-limited way, wherein the separator material 112 is a composite semi-conductor ceramic, comprised of an insulating phase and a conducting phase.

The operating mechanism of current semi-conductor ceramics based on Sialon and SiC relies on the intrinsic properties of one of the ceramic constituents (SiC surface conduction together with open porosity).

The composite ceramic is formed of a conductive phase included in an insulating matrix. This makes it possible to obtain an overall semi-conductor material with a microstructure of the same type as semi-conductor ceramic based on Sialon and SiC (grain boundaries, defects, etc.) and optimal thermomechanical properties.

The priming process thereby no longer depends on the intrinsic properties of one of the constituents and the microstructure of the surface, but on the material in its overall approach.

To prevent the injected charges from being led through the volume of the ceramic, the charges have to be implanted at a speed greater than the conduction speed, as reflected by the relaxation time that characterises the return to equilibrium after interruption of the field.

In the presence of conducting grains surrounded by insulating grain boundaries, the charges are trapped on the surface of the grains, thus inducing a curvature of the valence and conduction bands with the formation of a double Schottky barrier. The grain boundaries are indeed comparable to two back-to-back Schottky diodes. Trapping induces a space charge, so there is a field gradient and then a relaxation of this space charge, with this relaxation depending on the carrier transit time and the thickness of the insulating material. The greater the thickness of the insulating material, the greater the relaxation time and the higher the “breakdown” voltage (in fact conduction voltage).

Conduction in ceramics according to this example is controlled by several mechanisms:
first of all, at the interfaces of the ceramic: Schottky emission (the increase in electron emission when the applied electric field increases is due to the decrease in the extraction work), which determines injection quality and which is strongly correlated to the quality of the contacts and field emission,
and then, in the bulk of the ceramic: the Poole-Frenkel effect (trapping/untrapping) and the hopping effect (hop conduction).

The Fowler-Nordheim tunnel effect and the space charge also participate in conduction at the interfaces as well as in the bulk.

The restriction is now supported by the material ability to withstand severe environmental stresses.

Contemplatable materials for the conductive phase may be GaN, MoSi₂, HfB₂, TiB₂, ZrB₂ or even TiN.

The properties of these materials are given in the following table.

| | GaN | MoSi₂ | HfB₂ | TiB₂ | ZrB₂ | TiN |
|---|---|---|---|---|---|---|
| Vickers | 1 | 8700-9200 | | 3000 | 2300 | 2300 |
| Hardness | | | | | | |
| (MPa)/MOHS | | | | | | |
| Thermal | 1.3 | 90 | | | | |
| conductivity | | | | | | |
| (N · m⁻¹ · K⁻¹) | | | | | | |
| Coefficient of | 3.17 | 6.8 | 5-7 | 7.8 | 5.9 | 8-8.5 |
| thermal | | | | | | |
| expansion | | | | | | |
| 10⁻⁶ K⁻¹) | | | | | | |
| Resistivity | 2 · 10⁻⁶ | 2.2 · 10⁻⁵ | 10⁻⁵ | 7 · 10⁻⁶ | 6 · 10⁻⁶ | 50 · 10⁻⁶ |
| (Ω · cm) | | | | | | |
| Melting | 2500 | 2020 | 3380 | 3225 | 3245 | 2950 |
| temperature | | | | | | |
| (°C) | | | | | | |

Molybdenum disilicide (MoSi₂) can be an advantageous material for making the conductive phase because of its intrinsic qualities but also because of its ability to resist oxidation.

Advantages of using a MoSi₂ base for the conductive phase are that MoSi₂ has a high melting temperature (2030 °C), that MoSi₂ has a high resistance to oxidation up to 1600 °C, that MoSi₂ has a high thermal conductivity of 50 WMK, and that MoSi₂ is thermodynamically stable.

When two phases of different electrical conductivity are mixed, the resulting compound is likely to have a very wide range of conductivity values. The conductive grains of MoSi₂ behave like connections between capacitances whose dielectric is formed by TAl₂O₃.

Near the critical region, only a few percolating beam paths remain, with the role of the capacitors thus becoming very important. With a microstructure having big “packets” of alumina, which are the capacitors governing the conduction mechanism and which are too large to allow charges to pass therethrough by tunnel effect, charges remain trapped in the alumina and conduction remains very limited.

Conversely, when the conductive particles are small and, above all, well dispersed, the percolation threshold is lowered and there is instantaneous conduction as soon as a voltage is applied, as the inter-particle distance has been greatly reduced.

Two microstructures of the same composition can thus exhibit completely different behaviours depending on the arrangement of the two phases, and it is therefore necessary to have a compromise between the two extreme cases where the MoSi₂ grains percolate and where none are in contact.

Therefore, the microstructure according to the invention is a homogeneous distribution of MoSi₂ particles in the alumina matrix (by particle it is meant a grain or cluster of grains from 15 nm to 5 μm), this in order to allow charges to pass therethrough by tunnel effect, to compensate for the pulling out of too big MoSi₂ particles which would lead to breaking in the conduction process by distant percolation and to obtain satisfactory mechanical properties.

The presence of a “conductive particle size gradient” within the insulating matrix makes it possible to obtain the expected overall electrical effect (the material behaving as a totally insulating and then a conducting material). The particle size distribution is, for example, according to the following scheme:
“small particles”: mean diameter less than 240 nm (19.5 to 24% of the particles in the conductive phase);
“medium particles”: mean diameter from 240 nm to 1 μm (45.5 to 56% of the particles in the conductive phase);
“big particles”: mean diameter of 1 μm to 11 μm (20 to 35% of the particles in the conductive phase).

In order to avoid obtaining a conductive material as soon as the voltage is applied, it is preferable, for a same conductor/insulating ratio, to limit charges passing therethrough (no contact, no uniformly small inter-particle distances); this is why this particle size gradient is large.

In order to delay conduction, big particles are used, few in number and far apart from each other.

The probability of transfer by tunnel effect decreases with the interparticle distance; when there are big particles, it is preferable to compensate for these distances by defects in the interparticle zone or by the presence of small particles which ensure conduction still without contact but by tunnel effect made easier by the smaller interparticle distances.

The mean particles are of “intermediate” utility, they ensure conduction once it is established (and can play the role of either large or small particles according to where they are) and enable homogenisation of the material.

The expected microstructure is therefore grains as well as agglomerates of MoSi₂ grains (of controlled sizes) uniformly distributed in the matrix.

It will be noted that, when the size of the alumina grains is reduced, the number of grain boundaries is increased and the number of trapping regions is thus extended, increasing conduction; the size of the alumina grains is therefore also a factor to be controlled.

A semiconductor ceramic is therefore provided including:
5 to 40% by volume of a particulate conducting phase, preferably based on MoSi₂ particles;
60 to 95% by volume of a particulate insulating phase, the particle size of the conducting phase being between 5 nm and 10 μm, and the distance between two neighbouring particles of the conducting phase being between 0.1 and 10 μm.

Preferably, a semiconductor ceramic is provided including:
10 to 30% by volume of a conductive phase based on MoSi₂ particles,
70 to 90% by volume of a particulate insulating phase, the size of the MoSi₂ particles being between 15 nm and 5 μm, and the distance between two neighbouring MoSi₂ particles being between 0.1 and 6 μm.

Within the scope of the present invention, by “particle”, it is meant a grain or an aggregate/cluster of grains.

The measurement method used to measure the size of the MoSi₂ particles comprises observation under a scanning electron microscope of the rupture appearance of broken samples.

The images are then reprocessed using ESIVISION AnalySIS 3.2 software. The mean diameter of the MoSi₂ particle is obtained.

Advantageously, the semiconductor ceramic can include 15 to 25% by volume of MoSi₂ and preferably 21 to 24% by volume of MoSi₂.

By a “conductive phase based on MoSi₂ particles”, it is meant, according to the present invention, as being essentially made up of these particles. However, it may comprise other constituents such as carbon, boron or different metals.

Preferentially, the conductive phase according to the invention comprises more than 90% of MoSi₂ particles, more preferentially more than 95%, even more preferentially more than 97% of MoSi₂.

In an alternative embodiment of the ceramic according to the invention, the MoSi₂ conducting phase comprises between 0.1% and 3% by mass (of the total mass of the insulating phase+conducting phase) of a simple element (C, B, etc.) or a rare earth. One advantage of introducing carbon (boron or rare earth) is that it improves the mechanical properties of the MoSi₂ conducting phase.

The insulating phase can be made based on Al₂O₃, Si₃N₄, mullite (2 SiO₂, 3 Al₂O₃), or even ALON.

Properties of these materials are given in the following table.

| | Al₂O₃ | Si₃N₄ | Mullite | ALON |
|---|---|---|---|---|
| Mohs/Vickers | 9 | 1580 | | 7.5 |
| Hardness | | | | |
| (MPa) | | | | |
| Thermal | 26-35 | 15-43 | 3-5 | 2.5 |
| conductivity | | | | |
| (N · m⁻¹ · K⁻¹) | | | | |
| Thermal | 6.7-9 | 3 | 5-6 | 2-3 |
| expansion | | | | |
| coefficient 10⁻⁶ | | | | |
| K⁻¹) | | | | |
| Resistivity | 1014 | 1012-1015 | 1010-1013 | 1011-1014 |
| (Ω · cm) | | | | |
| Melting | 2050 | 1900 | 1880 | 1470 |
| temperature | | | | |
| (°C) | | | | |

In one embodiment of the ceramic according to the invention, the insulating phase of the ceramic is made based on Si₃N₄. Indeed, this material has greater hardness, greater thermal conductivity and greater resistivity than Al₂O₃, mullite and ALON.

In another embodiment of the ceramic according to the invention, the insulating phase is made based on Al₂O₃.

Indeed, alumina (Al₂O₃) has a high electrical resistivity, excellent creep endurance at temperatures above 1400 °C and good resistance to chemical attack. Furthermore, one advantage of a composite of MoSi₂ and Al₂O₃ for the conductive and insulating phases of the ceramic is that these two materials have similar expansion coefficients, so as to reduce thermal stresses between them.

Advantageously, the particle size of the insulating phase can be between 0.3 and 3 μm.

The measurement method used to measure the particle size in the insulating phase comprises observation under a scanning electron microscope of the rupture appearance of broken samples. Images are then reprocessed using ESIVISION AnalySIS 3.2 software. The mean particle diameter in the insulating phase is obtained.

The separator material may have some porosity. This porosity is open porosity, with the pores accessible from the outer face of the material. This porosity in the material has the advantage of improving performance of the device. This porosity is preferably greater than or equal to 10%. Preferably this porosity is between 10% and 30%. It can also be contemplated that porosity is zero or small.

This porosity value is a function of the technique employed to make the ceramic. For example, if natural sintering is used to manufacture the ceramic, the mean porosity is less than or equal to 10%. If hot press (HP) sintering is used, the mean porosity of the ceramic is less than or equal to 2%. Finally, if Spark Plasma Sintering (SPS) is used, the mean porosity of the ceramic is less than or equal to 3%. All these sintering techniques will be described in more detail below.

However, in the hypothesis where the ceramic comprises open porosities, the surface of the semi-conductor ceramic can be vitrified. This makes it possible to minimise so-called “Pest” phenomenon which can lead to the disintegration of the semi-conductor ceramic. The vitrification of the surface of the semi-conductor ceramic therefore makes it possible to improve solidity of the ceramic.

According to one alternative for minimising the Pest phenomenon, the MoSi₂ conductive phase may comprise 1% by weight of an element selected from Al, Ta, Ti, Zr, Y and B. Advantageously, the semiconductor ceramic according to the invention may also comprise 0.1 to 0.9% by weight of a lanthanide compound (for example La₂O₃, or even La₂B₆). A lanthanide (La₂O₃, LaB₆) is a material that promotes electron emission. Thus, adding it in small amounts to the semiconductor ceramic makes it possible to enhance thermo-emission of the semiconductor ceramic.

A method for manufacturing semiconductor ceramic according to this example is now described.

In order to resolve problems of reliability over time of electroconductive ceramics, it is important to develop a material under optimal conditions and to manage its microstructure in order to obtain the desired properties. The electrical characteristics depend on the volume percentage of the conductive phase and the type of microstructure developed after sintering.

The generation method, whose most important parameters determine the microstructure and thus the physical characteristics of the sintered samples, is in fact the most important phase. These parameters especially relate to:
the nature of the powders,
the proportions of the insulating and conducting phases,
the amount and composition of the additives,
the deagglomeration processes,
the shaping technique,
the sintering conditions.

The method for manufacturing a semi-conductor ceramic comprises the following steps of:
preparing a homogeneous suspension of a conducting phase, for example based on MoSi₂ particles, to obtain a first slurry,
preparing a homogeneous suspension of a particulate insulating phase to obtain a second slurry,
mixing the first and second slurries to obtain a mixture of the two phases in the proportions sought (i.e. the first slurry representing 5 to 40% by volume of the mixture and the second slurry representing 60 to 95% by volume of the mixture),
drying and sieving the composition;
sintering of the composition to obtain a ceramic in which the particle size of the conducting phase is between 5 nm and 10 μm, and the distance between two neighbouring conducting phase particles is between 30 Å and 5 μm.

As previously described, the conducting phase may be selected from the group consisting of MoSi₂, TiB₂, TiN.

In addition, the insulating phase can be based on alumina Al₂O₃ or mullite, Si₃N₄. To prepare the first slurry, the MoSi₂ conductive phase is mixed with water with a pH between 8 and 10.

This improves homogeneity of the microstructure. To prepare the second slurry, the insulating phase is mixed with water with a pH of 10 containing a dispersant, advantageously a surfactant polymer of the ammonium polymethacrylate type such as DARVAN® C marketed by VANDERBILT. This dispersant prevents the agglomeration of the particles of alumina Al₂O₃. It is advantageously introduced at a level of 0.1% by weight.

The two slurries are then mixed to obtain a mixture of the two phases. The two slurries are thoroughly mixed using a jar rotator or ball mill. Milling lasts between 5 and 24 hours.

The mixture is then dried in an oven for 48 hours. In order to avoid differential sedimentation of the two types of particles, rapid drying by ROTOVAP may be contemplated.

The powder obtained in this way is milled in a mortar; and then placed with glass beads (10 mm diameter) in a 250 μm mesh sieve of an electric sieving machine, the powder coming through 250 μm is poured into a 100 μm mesh sieve containing alumina beads (3 mm diameter).

The composite powder thus collected and coarsely granulated is ready to be shaped, and then sintered. The shaping step is optional. Its implementation depends on the sintering technology used. The shaping step transforms the material into a raw product with a controlled size, shape and surface, and a particular density and microstructure. Careful control of the density and microstructure of a green ceramic is necessary to achieve the performance of the final product because defects introduced by the shaping method are generally not removable by sintering.

A smooth and regular surface is normally desirable and may be essential for some products. The strength should be sufficient to carry out operations that follow shaping. The reproducibility of the product is very important for industrial production. The size and density of the raw piece should be controlled in order to maintain a constant shrinkage factor between the green and sintered piece.

For the shaping step, so-called compaction or dry compaction technology can be used.

Compaction is a method for shaping powder or granulated material enclosed in a rigid or flexible mould.

Dry compaction is a method widely used because of its reproducibility and its ability to produce large format pieces of different shapes that do not exhibit shrinkage on drying. It preferably comprises the following steps:
(1) filling the matrix;
(2) compaction and shaping;
(3) ejecting the piece;
(4) densification.

The composition is then sintered. Unlike ceramic firing, sintering does not, in principle, involve bonding of particles through a glassy phase. The pressed powders become coherent and denser as a result of transformations affecting the surface of the particles and leading to solid-solid interfaces referred to as grain boundaries. The sintering conditions determine the microstructure and therefore the properties of the final material.

Advantageously, different technologies can be used for sintering the composition. For example, in one embodiment of the method, sintering is natural sintering. In another embodiment, the sintering is hot press sintering. In yet another embodiment, sintering is Spark Plasma Sintering (SPS).

For the implementation of hot press sintering, the press furnace used is of the Goliath type (Stein Heurtey Physitherm) combining a press (maximum load 20 tonnes) and a graphite resistance furnace (maximum temperature 2200 °C) whose use requires an inert atmosphere or a high vacuum.

Temperature control is ensured by a 5/26% tungsten/rhenium thermocouple placed in proximity to the resistor as well as by a bichromatic pyrometer (IRCON®) which measures the actual temperature on the surface of the tooling. Hot press sintering (HP) is used to obtain 37 mm pellets. It does not require any shaping of the pellets.

Optimal densification of the ceramic is achieved for a temperature between 1600 °C and 1700 °C (and preferably equal to 1650 °C) and a load of 45 MPa. The temperature at which the best densification/microstructure compromise favourable to the expected electrical behaviour is achieved is 1500 °C.

For the implementation of natural sintering, the same furnace as for hot pressing is used. Simply, in this case the samples are placed after the shaping step in a graphite crucible, and are not subjected to a load during sintering.

In order to limit gas exchange with the outside, the preformed pellets are placed in an alumina crucible for “bur baking”, i.e. using uncompacted powder of the same nature as the pellets and lining them to prevent any contact with the furnace atmosphere. It is also possible to perform a Spark Plasma Sintering (SPS), as described previously. Spark Plasma Sintering (SPS) makes it possible to densify materials by retaining the characteristics of the initial powders and to densify difficult materials.

SPS is a method similar to conventional hot pressing.

Precursors (metals, ceramics, polymers and their composites, etc.) are placed in an enclosure of graphite that allows uniaxial pressure to be applied during sintering.

The major difference in this method is that the heat source is not external. An electric current (direct current—pulsed direct current or alternating current) passes through the conductive pressing enclosure and also, in appropriate cases, through the sample.

Thus, the enclosure itself acts as a heating source, thereby achieving high heating rates (up to 600 °C/min and more) and good heat transfer to the sample.

Very compact sintered objects can be obtained at lower temperatures (a few hundred degrees less) and, above all, with significantly shorter sintering times (a few minutes) than with conventional methods.

Spark Plasma Sintering (SPS) is an extremely promising technique for improving shaping of existing materials.

Densification is increased by the use of a current or a pulsed field.

### Particular Examples of Assemblies 11

Assembly 11 is now described in more detail according to several exemplary embodiments, with reference to FIGS. 4 to 6.

The conventional geometries of assemblies 11 used in DBD devices can be used for device 1 with the separator material 112.

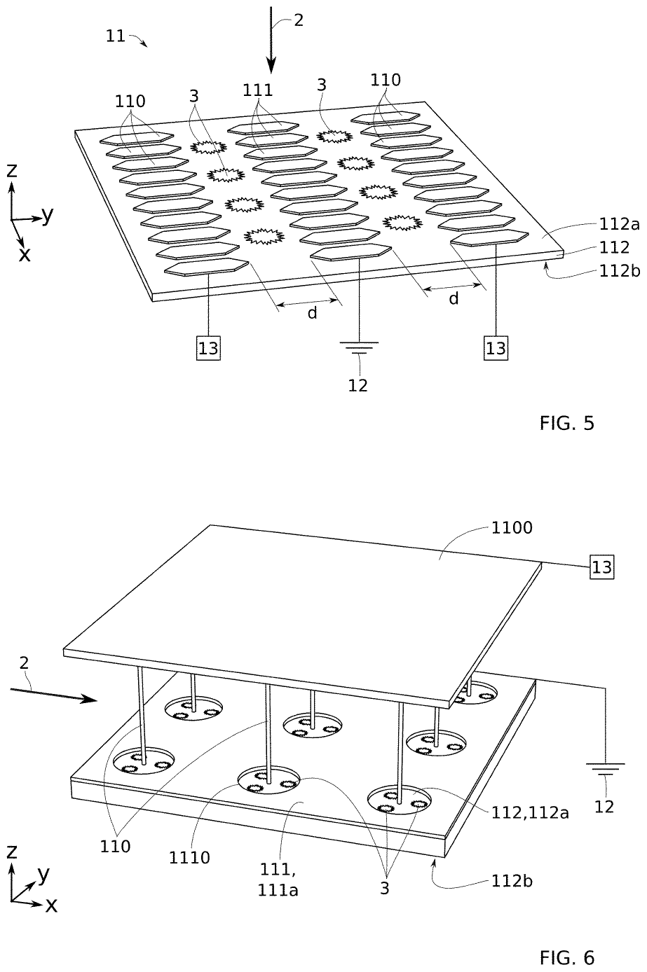

In addition, particular geometries have been developed. According to one example, at least the injection electrode 110 has a point configuration over at least one portion of the assembly 11. By an electrode with a “point configuration”, it is meant a configuration in which the electrode forms one or more peak structures, the tip-shaped end or equivalently peak-shaped end of these structures being disposed facing the other electrode, for example the electrode connected to the ground 111. The term “point” or “peak” does not necessarily imply that the end is conical; it may be sharp and planar. For example, the electrode may have a bottle brush structure, the ends of the bottle brush being disposed in line of sight of the other electrode, as illustrated in FIG. 4. According to another example, the electrode can extend in a main direction of extension between two point-shaped ends, as illustrated in FIG. 5. According to another example, the electrode can be in the form of a rod, the ends of the rod forming the point structures described above.

The point configuration of an electrode promotes plasma discharges in the erratic regime, in synergy with the nature of the separator material 112.

An initial geometry is described with reference to FIG. 4, according to an exemplary embodiment. The assembly 11 extends in a main direction of extension x substantially parallel to the direction of flow of the gas phase 2 in the assembly 11. The separator material 112 may have a cylindrical shape centred on the main direction of extension x, and separate the injection electrode 110 from the electrode connected to the ground 111. By “cylindrical” it is meant that the transverse cross-section of the separator material 112 may have a substantially circular, elliptical or ovoid shape.

The assembly 11 may have a first portion 11a and a second portion 11b, these portions being distinct from each other. Preferably, the first portion 11a is located upstream of the second portion 11b along the flow direction of the gaseous phase 2 in the assembly 11.

In the first portion 11a, the electrode connected to the ground 111 and the injection electrode 110 can form together a coaxial structure centred about the main direction of extension x, on either side of the cylindrical separator material 112. It is noted that the injection electrode 110 can be disposed inside the cylinder formed by the separator material 112, the electrode connected to the ground 111 then being disposed outside the cylinder, or vice versa. This first portion 11a promotes plasma discharges 3 in the homogeneous regime.

In the second portion 11b, the electrode connected to the ground can extend coaxially to the main direction of extension x about the separator material 112. The electrode connected to the ground 111 is then preferably disposed outside the separator material 112. The separator material 112 can delimit an internal volume 1120. The injection electrode 110 can be disposed in this internal volume 1120. The injection electrode 110 has a point configuration in this volume. For example, the injection electrode 110 may have a bottle brush-type structure whose point ends are disposed facing the separator material 112 and the electrode connected to the ground 111. Preferably, the point ends are disposed radially facing the separator material 112, about the main direction of extension x. This second portion 11b promotes generation of plasma discharges in the energetic regime.

In the first and second portions 11a, 11b, the electrode connected to the ground 111 can be in the form of a grid or a plate. In the first portion 11a, the injection electrode 110 can be in the form of a spiral.

Since the two portions 11a, 11b are distinct, it is understood that two treatment zones are formed as follows:
a first zone in which homogeneous plasma discharges 3 are promoted,
a second zone in which energetic plasma discharges 3 are promoted.

This geometry is particularly adapted for the degradation of some VOCs, such as sulphides, which are better degraded by a homogeneous plasma in the first zone. These zones are spatially distinct. Preferably, the gas phase 2 flows in the first zone and then in the second zone to be successively treated by a homogeneous plasma and then an energetic plasma.

As illustrated in FIG. 4, portions 11a, 11b may be separated by an intermediate portion 11c. Preferably, no plasma is generated in the intermediate portion 11c.

This intermediate portion 11c serves as a buffer between the plasma generation zones 3. The species generated in the first zone can thus react with each other to continue their degradation before entering the second zone.

Especially, ozone can react with reaction by-products or species 20 that have not been degraded in the first zone for their degradation.

Naturally, a person skilled in the art will be able to adapt geometry and dimensions as a function of the final application, and in particular as a function of the contact time of the plasma with the targeted species, the nature of the targeted species, the flow rate or volume, etc.

A second geometry and a third geometry are now described with reference to FIGS. 5 and 6 respectively. In these two geometries, the separator material 112 has a first face 11a and a second face 112b. The electrodes 110, 111 are disposed on a same face of the separator material 112, at a distance from each other. Electrodes 110, 111 can be disposed on each of the faces of the separator material 112. Thus, compactness of the device 1 is improved.

According to one example, the injection electrode 110 can have a point configuration facing the electrode connected to the ground 111. These geometries enable the generation of plasma discharges according to both homogeneous and energetic regimes. These two regimes can especially coexist spatially. The assembly thus has a treatment zone in which the two regimes coexist and follow each other temporally. In these two geometries, the energetic regime of plasma discharge 3 can be predominant.

In the second geometry, the injection electrode 110 and the electrode connected to the ground 111 each have a point configuration facing each other.

For example, each electrode 110, 111 can extend along one main direction of extension, for example the direction y, between two point-shaped ends. The electrodes 110 111 are disposed so that at least one end of each injection electrode 110 is facing one end of an electrode connected to the ground 111.

The assembly 11 can comprise several injection electrodes 110 and several electrodes connected to the ground 111. The injection electrodes 110 can be disposed in one or more rows along a direction substantially parallel to the direction x of flow of the gas phase 2 in the assembly 11. The electrodes connected to the ground 111 can also be disposed in one or more rows along a direction substantially parallel to the direction x of flow of the gas phase 2 in the assembly 11. A row of injection electrodes 110 can be spaced a distance d from a row of electrodes connected to the ground 111. The distance d can be selected to promote a particular plasma discharge regime. This is especially the case when the assembly 11 is supplied with a pulsed signal, as described in more detail subsequently. The distance d is, for example, between 3 and 25 mm, preferably between 10 and 15 mm.

In the third geometry, the separator material 112 can be at least partly covered with the electrode connected to the ground 111. The electrode connected to the ground 111 then has an upper face 111a comprising openings 1110 leaving the separator material 112 apparent.

The injection electrode 110 can extend from the opening 1110. The injection electrode 110 preferably extends from a first face 112a of the separator material 112, while being in contact with it. The injection electrode 110 can extend in an oblique direction, and preferably perpendicular, to the first face 111a of the electrode connected to the ground 111.

According to one example, the electrode connected to the ground 111 has several openings 1110, and an injection electrode 110 is disposed at each opening 1110.

The injection electrode 110 may be in the form of a rod, a first end of which is mounted to, preferably directly to, the separator material 112. The second end of the injection electrode 110 may be connected, preferably directly, to the power supply module 13.

In each of the geometries described, the injection electrode 110 can be connected directly or via another element, such as another injection electrode 110 or a conductive element, to the power supply module 13.

For each of the geometries described, the power supply module 13 configured to supply the assembly 11 can supply it with a pulsed electric current. This pulsed electric current comprises phases of application of the current interspersed with phases during which no current is applied. The electric signal supplying the assembly 11 is therefore discontinuous. The voltage applied to the assembly is therefore pulsed and may be positive or negative. A current application phase, or preferably each current application phase, may have a duration less than or equal to 1 μs, for example substantially equal to 500 ns. An pulsed electric current has the advantage of being able to more easily modulate the nature of the plasma discharge regime obtained.

For the second and third geometries, the power supply 13 can supply the assembly 11 with alternating electric current (abbreviated as AC). An AC power supply indeed requires that electrodes 110, 111 be disposed on the same face of the separator material 112. This type of power supply is cheaper and simpler to implement, which simplifies the design of device 1 and reduces its cost. An AC electric signal further allows the voltage to be applied to obtain a plasma discharge to be lowered even further. For example, the AC electric signal has a frequency of 50 Hz. By way of example, a plasma has been obtained with an alternating signal of 50 Hz at only 3 kV (relative to 20 kV for conventional DBD devices), with an inter-electrode distance of 10 to 15 mm for the second geometry.

It is noted that device 1 can be configured to be supplied with both pulsed and alternating current. This can make it easier, especially, to separate spatially one or more homogeneous plasma discharge zones from one or more energetic plasma discharge zones. For this, device 1 may comprise several assemblies 11, part of these assemblies 11 being supplied with pulsed current and the other part being supplied with alternating current. Alternatively or complementarily, for an assembly 11, and especially for the second and third geometries, part of the injection electrodes 110 can be supplied with pulsed current and the other part can be supplied with alternating current.

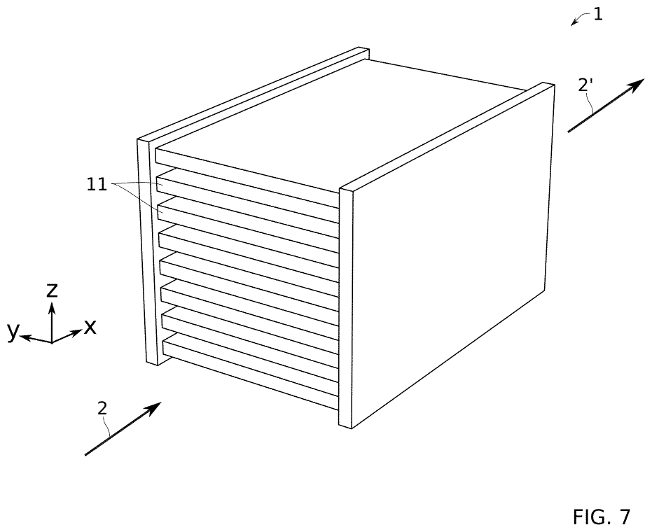

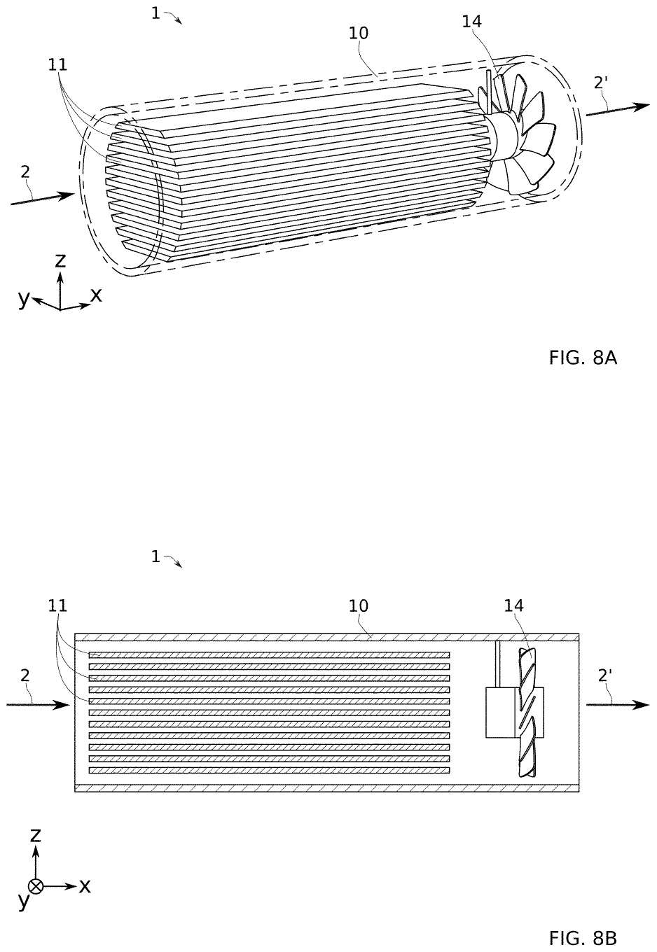

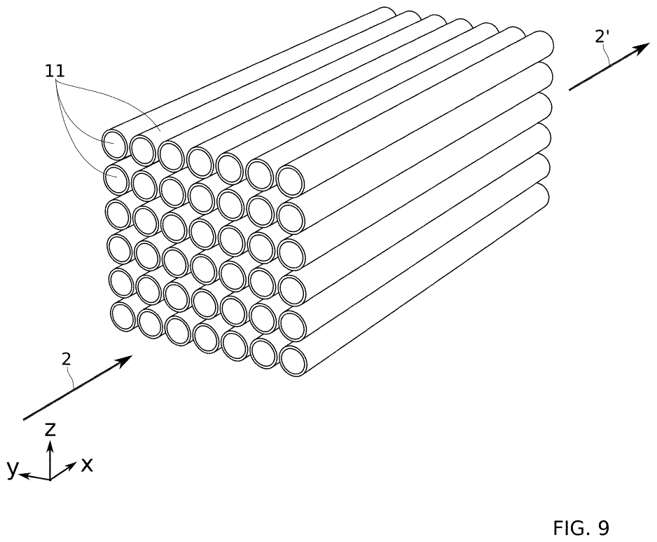

The device 1 preferably comprises several assemblies 11. These assemblies can be stacked in order to maximise the number of assemblies 11 and reduce the total volume of the device 1, in order to minimise its overall size. For this, the assemblies 11 can be stacked along a direction perpendicular to their main direction of extension x, for example direction z or direction y, or along two directions y, z perpendicular to direction x. As illustrated in FIGS. 7, 8A and 8B, the assemblies with planar geometry can be stacked along direction z, for example. Device 1 can thus have a rectangular shape as illustrated in FIG. 7, or a cylindrical shape as illustrated in FIGS. 8A and 8B. As illustrated in FIG. 9, cylindrical assemblies can be stacked along directions y and z to form a compact stack.

The device 1 can comprise assemblies 11 of different geometries, according to the overall size, the flow rate of the gas phase 2 to be treated, and/or even the type of pollution to be treated.

### Examples of VOC Treatments

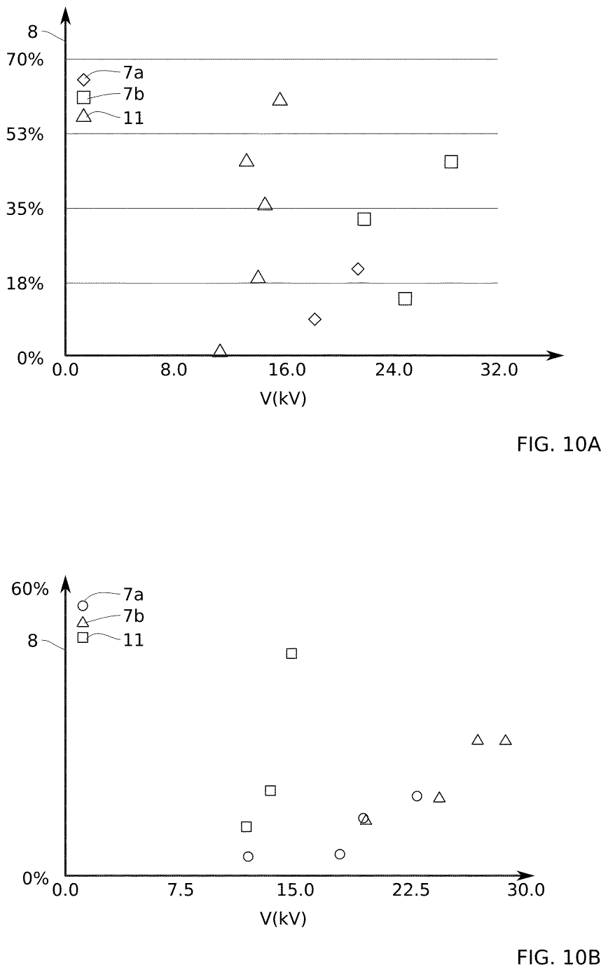

By way of example, the degradation of two VOCs has been measured with an assembly 11 with a conventional tip/plane geometry, therefore in terms of volume, with an inter-electrode distance of 4 mm. The applied signal is a positive square pulsed signal. The separator material 112 is a semiconductor ceramic according to the particular example described previously. These measurements have been compared with an assembly of a conventional DBD reactor comprising either an alumina separator material 7a or a glass separator material 7b. These graphs represent the destruction percentage 8 of VOC as a function of the voltage applied to generate the plasma (V in kV). In FIG. 10A, the gas phase 2 comprises ethylene at an initial concentration of 500 ppm. In FIG. 10B, the gas phase 2 comprises isopropanol at an initial concentration of 500 ppm. In both cases, VOCs are degraded at a voltage lower than that required to generate plasma for conventional DBD device assemblies. Furthermore, higher percentages of degradation are achieved for these two VOCs for assembly 11 comprising the semiconductor ceramic.

The assembly 11 described above has been tested on numerous other VOCs, for example, a gas phase containing 3000 ppm of a mixture of 32 sulphur compounds has been treated, which is representative of a very high concentration. A total VOC degradation rate of 75% has been achieved.

In view of the preceding description, it clearly appears that the invention provides a device that improves the treatment of a gas phase by plasma, especially air.

The invention is not limited to the embodiments described previously and extends to all embodiments covered by the invention. The present invention is not limited to the examples described previously. Many other alternative embodiments are possible, for example by combining previously described characteristics, without departing from the scope of the invention. Further, characteristics described in relation to one aspect of the invention can be combined with another aspect of the invention. Especially, the device may comprise any characteristic allowing implementation of a step of the method and the method may comprise any step resulting from the implementation of a characteristic of the device.

1. A device for treating a gaseous phase comprising at least one assembly of electrodes comprising at least one injection electrode and at least one electrode connected to a ground of the device, separated by a separator material, the at least one assembly being configured so that, under an application of an electric voltage, a plasma is generated between the at least one injection electrode and the at least one electrode connected to the ground,
and wherein the separator material exhibits electrically insulating behaviour when the separator material is subjected to an electric voltage below a threshold primer voltage, and electrically conductive behaviour allowing a current to flow therethrough when the separator material is subjected to an electric voltage greater than or equal to the primer voltage,
and wherein the separator material is a composite material comprising conductive or semiconductive particles dispersed in an insulating matrix, the insulating matrix being based on or made of a material selected from the group consisting of a polymer and a ceramic, and the conductive or semiconductive particles being based on or made of a material selected from the group consisting of a metal, an intermetallic compound, a metal alloy, a ceramic.

2. The device according to claim 1, wherein the injection electrode has a point configuration over at least one portion of the assembly.

3. The device according to claim 2, wherein the separator material has a cylindrical shape extending along a main direction of extension of the assembly, and:
on a first portion of the assembly, the electrode connected to the ground and the injection electrode together form a coaxial structure on either side of the separator material, about the main direction of extension of the assembly,
on a second portion of the assembly, distinct from the first portion, the electrode connected to the ground extends coaxially to the main direction of extension of the assembly about the separator material, and the injection electrode has a point configuration disposed in an internal volume defined by the separator material.

4. The device according to claim 3, wherein the first portion and the second portion are separated by an intermediate portion, the at least one assembly being configured such that, under the application of an electric voltage, a plasma is generated only in the first and second portions.

5. The device according to claim 2, wherein the separator material has a first face and a second face opposite to the first face, the injection electrode and the electrode connected to the ground are disposed on the first face of the separator material, the injection electrode and the electrode connected to the ground being disposed at a distance from each other.

6. The device according to claim 5, wherein, on the second face of the separator material, at least one injection electrode and at least one electrode connected to the ground are further disposed at a distance from each other.

7. The device according to claim 1, wherein the injection electrode and the electrode connected to the ground each have a point configuration facing between the injection electrode and the electrode connected to the ground.

8. The device according to claim 5, wherein the electrode connected to the ground has a first face comprising at least one opening leaving the separator material apparent, at least one injection electrode being disposed in the at least one opening and extending from the separator material along a direction oblique to the first face of the electrode connected to the ground.

9. The device according to claim 1, wherein the assembly is connected to a power supply module configured to supply the assembly with a pulsed electric signal.

10. The device according to claim 5, wherein the assembly is connected to a power supply module configured to supply the assembly with an alternating electric signal.

11. The device according to claim 1, wherein the separator material has a non-zero average porosity.

12. The device according to claim 1, comprising a plurality of assemblies stacked along at least one direction perpendicular to a main direction of extension of the assembly.

13. A method for treating a gas phase using the device according to claim 1, and comprising:
introducing the gaseous phase into the at least one assembly of electrodes,
applying an electric voltage to the assembly so as to generate a plasma between the at least one injection electrode and the at least one electrode connected to the ground to treat the gas phase.

14. The method according to claim 13, wherein the voltage applied to generate the plasma has a non-zero value of less than 10 kV.

15. The method according to claim 1, wherein, in the device, the gas phase is at atmospheric pressure.

16. The device according to claim 11, wherein the non-zero average porosity is greater than or equal to 10%.

17. The device according to claim 16, wherein the non-zero average porosity is between 10% and 30%.

18. The method according to claim 14, wherein the voltage applied to generate the plasma has a non-zero value of less than 6 kV.
